(19) United States
(12) Patent Application Publication
(10) Pub. No.: US 2020/0038689 A1
(43) Pub. Date: Feb. 6, 2020

(54) METHOD AND APPARATUS FOR PROVIDING FEEDBACK TO A PATIENT ON HIS OR HER BREATHING DURING RADIOTHERAPY

(71) Applicant: OPASCA GmbH, Mannheim (DE)

(72) Inventors: Timo Machmer, Neuhofen (DE); Alexej Swerdlow, Ludwigshafen (DE); Steffen Liebscher, Ludwigshafen (DE)

(21) Appl. No.: 16/477,086

(22) PCT Filed: Jan. 11, 2018
(86) PCT No.: PCT/DE2018/200000

§ 371 Date: Sep. 30, 2019

(30) Foreign Application Priority Data
Jan. 12, 2017 (DE) 10 2017 200 400.2

(51) Int. Cl. A61N 5/10

(52) U.S. Cl. CPC A61N 5/1068; A61N 5/1037; A61N 5/107

(57) ABSTRACT

A method for improved precision of irradiation in radiation therapy includes providing feedback to a patient with respect to the patient's respiration during the radiation therapy, wherein information about the patient's current respiratory state and/or instructions for action relating to the patient's respiration are communicated to the patient by means of ambient signals. Also described herein is a device for applying such a method.

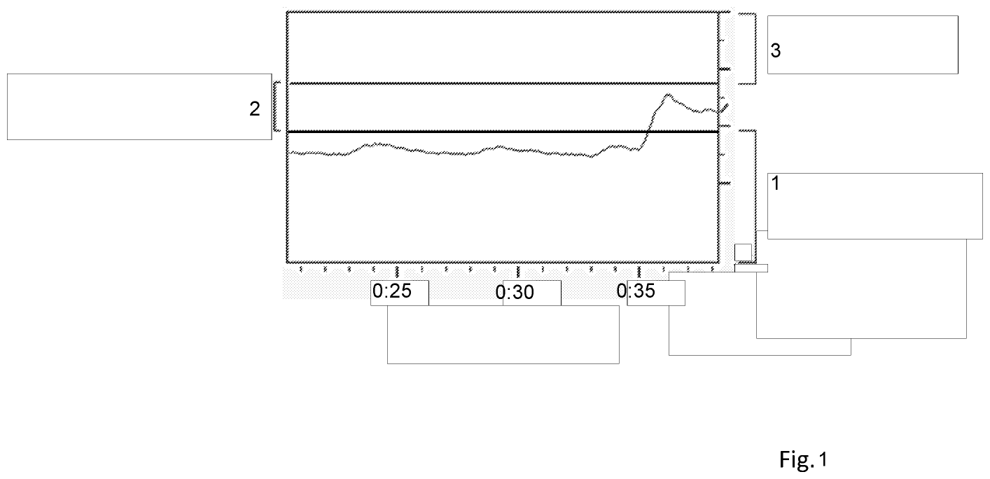

### BACKGROUND

### Technical Field

The present disclosure relates to a method and a device for providing feedback to a patient with respect to his respiration in radiation therapy.

### Description of the Related Art

During inhalation and exhalation, the organs in the chest and abdomen regions are mostly displaced during a breathing cycle as a result of the respiratory movements. This influences the accuracy of irradiation in radiation therapy, as a result of which organs that are not to be irradiated, i.e., healthy organs, are irradiated.

In practice, respiratory-dependent irradiation—so-called respiratory gating—is used in radiation therapy. In this method, the irradiation occurs in a specific respiratory state or a specific respiratory phase—a so-called safety range (gate). In most cases, this is a range of deep respiration in which the patient is supposed to hold his breath. This safety range can be determined by imaging methods—for example, computer tomography. By means of the respiratory gating method, the position of the tumor/tumor site to be irradiated can, on the one hand, be stabilized, and, on the other, the irradiation of healthy tissue and nearby organs can be reduced. For example, with breast cancer, the heart or, in the case of lung tumors, the healthy lobe of the lung can be spared during irradiation.

A major practical problem of the respiratory gating method in clinical use is providing the patient with timely and accurate feedback about his current respiratory state. In concrete terms, the patient must be given instructions as to whether he should inhale (further) or exhale (again) or whether his respiration is already in the safety range and he is to hold his breath for the irradiation.

The currently most frequent and simplest variant for informing the patient about his current respiratory state is based upon voice communication by the medical personnel administering the radiation therapy. The personnel give the patient voice commands in the form of “Please inhale more deeply,” “Hold your breath,” or “Exhale.” Significant disadvantages of this approach are both the time delay of the voice commands and the vagueness of the instructions for action due to the language. It is often difficult to achieve a smooth treatment procedure—in particular, in the case of foreign-language patients and/or personnel and/or in the case of strong dialects. In addition, the voice commands constitute additional effort for the personnel and compromise the concentration and focusing on other, treatment-specific information.

In practice, visual methods using virtual reality (VR) glasses or monitors in the treatment room represent an alternative to speech commands from the medical personnel. This type of feedback to the patient is based upon schematic displays of his current respiratory state and the safety range. The disadvantage with the use of VR glasses is the additional effort in handling and the prescribed disinfection of the glasses after the treatment of each patient. Disadvantageous in the case of the monitors is usually an unfavorable viewing angle of the monitor due to the position of the patient on the irradiation table. Patients' visual impairments constitute a further obstacle in both of the above-mentioned visual methods with schematic displays.

### BRIEF SUMMARY AND GENERAL DESCRIPTION

The aim of the present disclosure is therefore to provide a possibility, with little demand upon medical personnel, of conveying to the patient timely and unambiguous signals regarding his respiratory state.

According to the disclosure, the above aim is accomplished by providing feedback to the patient with regard to his respiration in radiation therapy, in which information about his current respiratory state and/or instructions for action with respect to his respiration are communicated to the patient by means of ambient signals, preferably as part of respiratory gating.

In the manner according to the disclosure, it has first been recognized that it is not necessary for medical personnel to be occupied in giving the patient voice commands and/or to struggle with voice and/or acoustic hurdles. It has also been recognized that it is of particular advantage not to let the patient read information and instructions via schematic displays on a monitor or VR glasses and to manage these devices for this purpose. By contrast, the present disclosure takes a completely different path, viz., information about the patient's current respiratory state and/or instructions for action regarding his respiration is communicated to him via unambiguous, timely ambient signals. Due to this type of feedback, a time delay in the commands and linguistic obstacles no longer occur, the personnel are able to concentrate on other treatment-specific information, the patient simply and unambiguously recognizes the information and instructions for action communicated to him with respect to his respiration, and unnecessary work steps, such as disinfection of VR glasses and/or an adjustment of a monitor, are not required. An unfavorable position of the patient on the treatment table is also thus irrelevant.

A device according to the present disclosure for improved accuracy of the irradiation in radiation therapy applies a method as described above. The device comprises means for generating ambient signals which convey information to the patient about his current respiratory state and/or instructions for action with respect to his respiration.

Consequently, with the claimed method and with the claimed device, a possibility is provided with which, with little effort on the part of medical personnel, timely and unambiguous signals regarding his respiratory state can be communicated to the patient.

The ambient signals can essentially be considered a change in the spatial ambience. The ambient signals are, advantageously, ambient lighting—in particular, ambient color(s)—and/or a change in the brightness of an ambient lighting. Alternatively or additionally, ambient sound is conceivable—in particular, a tone sequence(s) and/or a change in the volume of the ambient sound. Based upon the respiratory state or the respiratory phase of the patient, the ambient lighting and/or the ambient sound are used to provide information about his current respiratory state and/or provide instructions for action with respect to his respiration. For this purpose, the ambient lighting or the ambient sound can be coded according to the respiratory state and/or according to the instructions for action. For example, reaching the safety range can be indicated by a change in the ambient illumination to green. In the case of ambient sound, it is conceivable to signal to the patient his current respiratory phase in an acoustically coded manner by means of corresponding modulation of a carrier signal. A tone sequence and/or a change in volume of the ambient sound are conceivable.

In a further advantageous manner, the ambient signals may signal the “Inhale” and/or “Hold your breath” and/or “Exhale” instructions for action.

Another difficulty with respiratory gating, however, is often that the patient does not have any feeling for how far away his current respiratory state is from the required safety range. This in turn leads to uncertainty on the part of the patient and makes treatment more difficult. In order to counteract this, it is helpful to signal the remaining duration of the inhalation, e.g., by color coding the difference from the required safety range, so that the patient recognizes an approach to the safety range by the ambient color.

In principle, it is important that the required dose of radiation be introduced into the target area—the tumor. The irradiation is emitted in a fixed dose—so-called monitor units per time. The result is that the application of different doses requires irradiation of different lengths. In the case of respiratory gating, for the patient, this means that, depending upon the dose, he must hold his breath in the safety range for a different length of time in order to enable the dose to be introduced in the target volume. In practice, no solutions are known today which signal to the patient how long he has to hold his breath. This also frequently leads to uncertainty and even nervousness in the patients, right up to agonal respiration, which considerably obstructs a smooth course of treatment. In order to avoid this and give the patient more security, and to ensure a more rapid and precise treatment, the duration of the breath-holding and/or of the exhalation can additionally or alternatively be signaled by ambient signals. This can be implemented, for example, by coding the time period still to be irradiated as ambient lighting and/or ambient sound.

It is conceivable that a safety range of the respiration—the so-called gate for protecting organs not to be irradiated—be determined by means of imaging methods—in particular, by means of computer tomography.

The ambient signals are, advantageously, based upon the current respiratory state. Optical determination of the current respiratory state—in particular, by camera systems for recording the patient's respiratory movement—and/or a mechanical determination—in particular, by strain gauges—is conceivable. This can preferably take place automatically. Automated methods for determining the patient's current respiratory state make it possible to track the “stroke” of the thorax relative to a reference plane throughout the entire radiation treatment.

Continuous monitoring of the gating process or of the respiration during the treatment ensures the detection of a plurality of parameters, in order to statistically evaluate individual, patient-typical respiratory behavior. In particular, the individual ability to hold one's breath can thus be determined. This ability is very different from patient to patient. This depends upon various factors, such as the physical condition and/or age of the patient.

Depending upon the required dose of radiation which has to be introduced during the treatment, phases of breath-holding and irradiation of different lengths are required. The individual, patient-typical respiratory behavior or the ability to hold one's breath results in the individual phases of the breath-holding differing in length according to the patient and/or a different number of repetitions of the breath-holding and irradiation having to be carried out. Particularly in connection with a system for indicating the irradiation time still pending, patients frequently feel under a lot of pressure, which can lead to a change in respiratory behavior right up to agonal respiration, and again complicates the treatment. By detecting and evaluating the individual, patient-typical respiratory behavior, the optimal time span of the breath-holding and/or the number of required repetitions of the breath-holding can, advantageously, be determined individually for each patient as a function of the required irradiation.

Existing statistics for a patient can, advantageously, be used to determine the duration of the breath-holding and/or the number of repetitions of the breath-holding. However, if there are no statistics or information for the patient about his breathing, it is conceivable that general—especially, age and/or gender-dependent—patient statistics be used to determine the duration of the breath-holding and/or the number of repetitions of the breath-holding. For example, phases of breath-holding that can be carried out to be individually suitable and pleasant for each patient, and thus time spans of irradiation, can be derived from collected statistics for each patient by means of scientific algorithms.

In order to ensure a particularly flexible and efficient treatment, it is conceivable for the treatment personnel to be able to carry out a fine adjustment of the time span of the breath-holding and/or the number of repetitions of the breath-holding, and thus of the irradiation, during the treatment. For this, an indication of the proposed time periods at the operating site is advantageous.

There are various options for advantageously designing and developing the teaching of the present disclosure. To this end, reference is made, on one hand, to the claims subordinate to claim 1 and, on the other, to the subsequent explanation of a preferred exemplary embodiment of the disclosure based upon the drawing. In connection with the explanation of the preferred exemplary embodiments of the disclosure based upon the drawing, generally preferred designs and developments of the teaching are also explained.

### BRIEF DESCRIPTION OF THE DRAWING

The FIGURE shows a schematic diagram of a respiratory curve during respiratory gating in radiation therapy.

### DETAILED DESCRIPTION

The single FIGURE shows in a schematic diagram a respiratory curve during respiratory gating in radiation therapy, wherein information about the patient's current respiratory state and instructions for action with respect to his respiration are communicated to the patient by ambient signals, in this case in the form of ambient colors. The horizontal axis of the diagram shows the time course, and the vertical axis shows the depth of respiration of the patient. The area with reference numeral 1 is a range of shallow breathing. In this breathing phase, the ambient lighting is neutral—that is to say, white. This indicates to the patient that he has not yet reached the safety range—the gate—and has to inhale further. If he reaches the safety range 2, the ambient light switches to green, and the patient knows that he has to hold his breath for as long as the ambient lighting has a green color, so that the irradiation can take place. Should the patient have inhaled too deeply 3, the ambient light switches to orange, signaling to the patient that his breathing is too deep, and he is to exhale again.

The safety range of the respiration for protecting organs not to be irradiated was determined beforehand by means of computer tomography. The change in the ambient colors is based upon the current, automatically detected respiratory state of the patient. This is determined optically by means of a camera system for recording the respiratory movement. During the treatment, the individual, patient-typical respiratory behavior—in particular, the individual ability to hold one's breath—is detected and analyzed statistically by continuously monitoring the respiration. This makes it possible, inter alia, to determine the duration of the breath-holding and/or the number of required repetitions of the breath-holding as a function of the required irradiation. Fine adjustment of the time span of the breath-holding and/or the number of repetitions during the treatment by the medical personnel is also possible.

The preferred exemplary embodiment has the advantage that the feedback to the patient (patient feedback) takes place in an extremely timely manner and is easy for the patient to understand, regardless of linguistic barriers, visual impairments, etc. Moreover, unnecessary work steps for the personnel are dispensed with, and they can concentrate on other, treatment-specific activities and information.

With regard to other advantageous embodiments of the device according to the disclosure, to avoid repetition, reference is made to the general part of the description and also to the accompanying claims.

Finally, it is expressly pointed out that the above-described exemplary embodiments of the device according to the disclosure serve only to explain the claimed teaching, but do not limit it to the exemplary embodiments.

### LIST OF REFERENCE SYMBOLS

1 Range for shallow respiration, neutral color
2 Safety range, green color
3 Range for deep respiration, orange color

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for providing feedback to a patient with respect to the patient's respiration in radiation therapy, comprising:
communicating to the patient, by ambient signals, information about the patient's current respiratory state and/or instructions for action with respect to the patient's respiration as part of respiratory gating.

2. The method according to claim 1, wherein the ambient signals are ambient lighting, including ambient color(s) and/or a change in the brightness of ambient lighting.

3. The method according to claim 1, wherein the ambient signals signal an “Inhale” instruction for a remaining duration of inhalation and/or a “Hold your breath” instruction for breath-holding and/or an “Exhale” instruction for exhalation.

4. The method according to claim 1, wherein a safety range of respiration for protecting organs not be irradiated by the radiation therapy is determined by computer tomography.

5. The method according to claim 1, wherein the ambient signals are based upon a current, automatically detected respiratory state.

6. The method according to claim 5, wherein the current respiratory state is determined optically by at least one camera system.

7. The method according to claim 1, further comprising continuously monitoring the patient's respiration and based on said monitored respiration, detecting and statistically evaluating the patient's capability of holding his breath.

8. The method according to claim 1, further comprising, by detecting and evaluating an individual, patient-typical respiratory behavior of the patient, determining a duration of breath-holding and/or a number of required repetitions of breath-holding by the patient as a function of irradiation required by the radiation therapy.

9. The method according to claim 8, wherein existing statistics and/or general patient statistics dependent upon age and/or gender are used to determine the patient's duration of breath-holding and/or the number of required repetitions of breath-holding.

10. The method according to claim 8, further comprising finely adjusting the duration of breath-holding and/or the number of required repetitions of breath-holding during the radiation therapy.

11. A device for providing feedback to a patient regarding the patient's respiration in radiation therapy, in particular in respiratory gating, for application of the method according to claim 1, comprising means for generating the ambient signals which communicate information about the patient's current respiratory state and/or instructions for action with respect to the patient's respiration.

12. The method according to claim 1, wherein the ambient signals are ambient sound, including tone sequence(s) and/or a change in volume of the ambient sound.

13. The method according to claim 5, wherein the current respiratory state is determined mechanically by at least one strain gage.
